(19) United States
(12) Patent Application Publication
(10) Pub. No.: US 2014/0236166 A1
(43) Pub. Date: Aug. 21, 2014

(54) BIOMARKERS FOR DISTINGUISHING BENIGN, PRE-MALIGNANT, AND MALIGNANT PANCREATIC CYSTS

(71) Applicants: Walter G. Park, Mountain View, CA (US); Pankaj J. Pasricha, Columbia, MD (US); Gary Peltz, Palo Alto, CA (US); Anson Lowe, San Francisco, CA (US)

(72) Inventors: Walter G. Park, Mountain View, CA (US); Pankaj J. Pasricha, Columbia, MD (US); Gary Peltz, Palo Alto, CA (US); Anson Lowe, San Francisco, CA (US)

(73) Assignee: The Board of Trustees of the Leland Stanford Junior University, Palo Alto, CA (US)

(21) Appl. No.: 14/180,892

(22) Filed: Feb. 14, 2014

Related U.S. Application Data
(60) Provisional application No. 61/765,306, filed on Feb. 15, 2013.

(51) Int. Cl. G01N 33/574

(52) U.S. Cl. CPC G01N 33/57438
USPC 606/110; 435/7.94

(57) ABSTRACT

Methods for prognosis and diagnosis of pancreatic cysts are disclosed. In particular, the invention relates to the use of biomarkers from pancreatic cyst fluid to aid in the diagnosis, prognosis, and treatment of pancreatic cysts. More specifically, differential expression of certain metabolites, including glucose and kynurenine, and the protein, amphiregulin, is used to distinguish benign, pre-malignant, and malignant pancreatic cysts.

### CROSS-REFERENCE TO RELATED APPLICATION

This application claims benefit under 35 U.S.C. §119(e) of provisional application 61/765,306, filed Feb. 15, 2013, which is hereby incorporated by reference in its entirety.

### STATEMENT REGARDING FEDERALLY SPONSORED RESEARCH OR DEVELOPMENT

This invention was made with Government support under contracts DK090992 and DK063624 awarded by the National Institutes of Health. The Government has certain rights in this invention.

### TECHNICAL FIELD

The present invention pertains generally to biomarkers for use in diagnosis, prognosis, and treatment of pancreatic cysts. In particular, differential expression of certain biomarkers, including glucose, kynurenine, and amphiregulin, is used to distinguish benign, pre-malignant, and malignant pancreatic cysts.

### BACKGROUND

Pancreatic cysts are increasingly recognized from routine use of computed tomography and magnetic resonance imaging with current prevalence estimates of 2% in the population, rising to approximately 8% in the elderly (de Jong et al. (2010) Clin. Gastroenterol. Hepatol. 8(9):806-811; Laffan et al. (2008) AJR Am. J. Roentgenol. 191(3):802-807). Appropriate diagnosis and management of these cysts is clinically important because approximately half may have potential for malignant transformation to pancreatic adenocarcinoma—a cancer associated with an overall 5-year survival rate of 5% (Fernandez-del Castillo et al. (2003) Arch. Surg. 138(4):427-434, discussion 33-34; In SEER Cancer Statistics Review, 1975-2007, National Cancer Institute, Bethesda, Md. Edited by: Altekruse S, Kosary C L, Krapcho M, Neyman N, Aminou R, Waldron W, Ruhl J, Howlader N, Tatalovich Z, Cho H, Mariotto A, Eisner M P, Lewis D R, Cronin K, Chen H S, Feuer E J, Stinchcomb D G, Edwards B K, 2010). Cysts with malignant potential include mucinous cystic neoplasms (MCN) and intraductal papillary mucinous neoplasms (IPMN).

Various diagnostic tests, including endoscopic ultra-sound (EUS), are employed to facilitate diagnosis and management of pancreatic cysts (Brugge et al. (2004) N. Engl. J. Med. 351(12):1218-1226; Ahmad et al. (2001) Am. J. Gastroenterol. 96(12):3295-3300). EUS guided aspiration of cyst fluid provides an opportunity to evaluate for tumor markers such as carcinoembryonic antigen (CEA) that can differentiate mucinous from non-mucinous cysts with reasonable accuracy. CEA cannot, however, accurately differentiate pre-malignant cysts from malignant cysts (Brugge et al. (2004) Gastroenterology 126(5):1330-1336). Further, cyst fluid cytology also possesses low sensitivity for diagnosing malignancy (Jacobson et al. (2005) Gastrointest. Endosc. 61(3):363-370). Because progression to cancer may be slow and variable among pre-malignant mucinous cysts, biomarkers that identify cysts with cancer or high-grade dysplasia may have clinical value by identifying which patients may benefit from immediate consideration for surgery (Das et al. (2008) Am J Gastroenterol 103(7):1657-1662; Rautou et al. (2008) Clin. Gastroenterol. Hepatol. 6(7):807-814; Tanno et al. (2008) Gut 57(3):339-343; Kang et al. (2011) Clin. Gastroenterol. Hepatol. 9(1):87-93).

There remains a need in the art for improved methods for diagnosing pancreatic cysts that can distinguish benign and malignant pancreatic cysts in order to identify subjects at high risk of developing pancreatic cancer who are in need of surgical intervention.

### SUMMARY

The present invention relates to the use of biomarkers for aiding diagnosis, prognosis, and treatment of pancreatic cysts. The inventors have shown that monitoring levels of the metabolites, glucose and kynurenine, and the protein, amphiregulin, is useful in distinguishing mucinous and non-mucinous pancreatic cysts and for identifying patients with high risk of progression to pancreatic cancer (see Examples 1 and 2).

In the methods of the invention, a sample of pancreatic cyst fluid is collected from a subject, for example, by endoscopic ultrasound fine-needle aspiration or surgically, and the levels of one or more biomarkers selected from the group consisting of glucose, kynurenine, and amphiregulin are measured and compared with reference levels for the biomarkers in mucinous and non-mucinous pancreatic cysts. The reference levels can represent the amount of a biomarker found in one or more samples of one or more non-mucinous cysts. Alternatively, the reference levels can represent the amount of a biomarker found in one or more samples of one or more mucinous cysts. More specifically, the reference levels for a biomarker can represent the amount of a biomarker in a particular type of non-mucinous or mucinous pancreatic cyst (e.g., pseudocyst, serous cystadenoma, mucinous cystic neoplasm, or intraductal papillary mucinous neoplasm) to facilitate a determination of the type of pancreatic cyst present and the malignant potential of the pancreatic cyst in an individual.

In one embodiment, the level of glucose is measured and compared to reference levels for glucose in pancreatic cyst fluid of mucinous and non-mucinous pancreatic cysts. A level of glucose greater than or equal to 66 mg/dL indicates that a pancreatic cyst is a non-mucinous pancreatic cyst. A level of glucose less than 66 mg/dL indicates that a pancreatic cyst is a mucinous pancreatic cyst.

In another embodiment, the level of kynureinine is measured and compared to reference levels for kynureinine in pancreatic cyst fluid of mucinous and non-mucinous pancreatic cysts. A lower level of kynurenine in a sample of pancreatic cyst fluid from a subject compared to the level of kynureinine in pancreatic cyst fluid from one or more benign non-mucinous cysts indicates that the pancreatic cyst in the subject is a mucinous pancreatic cyst. In one embodiment, levels of both glucose and kynurenine are measured.

In another embodiment, the level of amphiregulin is measured, wherein a level of amphiregulin greater than 300 pg/ml in pancreatic cyst fluid indicates that a pancreatic cyst is a malignant mucinous pancreatic cyst. Additionally, the level of amphiregulin in pancreatic cyst fluid samples from a subject may be compared to reference levels for amphiregulin for high grade dysplasia, cancer in situ, and invasive cancer to determine the stage of disease progression in an individual.

The biomarkers can be measured by any suitable method including, but not limited to, mass spectrometry, an enzymatic or biochemical assay, liquid chromatography, NMR, an enzyme-linked immunosorbent assay (ELISA), a radioimmunoassay (RIA), an immunofluorescent assay (IFA), or a Western Blot. In one embodiment, the level of glucose is measured using a hexokinase-glucose-6-phosphate dehydrogenase coupled enzyme assay. In another embodiment, the level of amphiregulin is measured by contacting an antibody with amphiregulin, wherein the antibody specifically binds to amphiregulin, or a fragment thereof containing an antigenic determinant of amphiregulin. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments. In another embodiment, the biomarkers are detectably labeled and measured after separation by liquid chromatography. For example, the level of a biomarker can be determined from analysis of a chromatogram by integration of the peak area for the eluted biomarker.

In one aspect, the invention includes a method for distinguishing mucinous and non-mucinous cysts, the method comprising: a) obtaining a sample of pancreatic cyst fluid from a subject; b) measuring the levels of one or more biomarkers in the pancreatic cyst fluid, wherein the one or more biomarkers are selected from the group consisting of glucose and kynurenine; and c) analyzing the levels of one or more biomarkers in conjunction with respective reference levels for the biomarkers, wherein similarity of the levels of one or more biomarkers in the cyst fluid to reference value levels for a mucinous cyst indicates that the cyst in the subject is a mucinous cyst, and wherein similarity of the levels of one or more biomarkers in the cyst fluid to reference levels for a non-mucinous cyst indicates that the cyst in the subject is a non-mucinous cyst. In one embodiment, the method further comprises measuring the level of amphiregulin to distinguish malignant and non-malignant mucinous pancreatic cysts.

In another aspect, the invention includes a method of monitoring a pancreatic cyst in a subject, the method comprising: a) analyzing a first pancreatic cyst fluid sample from a subject to determine the levels of one or more biomarkers, wherein the one or more biomarkers are selected from the group consisting of glucose, kynurenine, and amphiregulin, wherein the first sample is obtained from the subject at a first time point; b) analyzing a second pancreatic cyst fluid sample from the subject to determine the levels of the one or more biomarkers, wherein the second sample is obtained from the subject at a second time point; and c) comparing the levels of the one or more biomarkers in the first pancreatic cyst fluid sample to the levels of the one or more biomarkers in the second pancreatic cyst fluid sample in order to detect any changes in the status of the pancreatic cyst in the subject over time.

In another aspect, the invention includes a method for treating a pancreatic cyst in a subject, the method comprising: obtaining a sample of pancreatic cyst fluid from the pancreatic cyst in the subject, and surgically removing the pancreatic cyst from the subject if the level of amphiregulin in the pancreatic cyst fluid sample is greater than 300 pg/ml.

In another aspect, the invention includes a method for determining the prognosis of a subject having a pancreatic cyst. The method comprises measuring the levels of one or more biomarkers in a pancreatic cyst fluid sample derived from the subject, wherein a level of glucose greater than or equal to 66 mg/dL indicates that the subject is at low risk of developing pancreatic cancer; and a level of amphiregulin greater than 300 pg/ml indicates that the subject is at high risk of developing pancreatic cancer.

In another aspect, the invention includes a method for monitoring the efficacy of a therapy for treating pancreatic cancer or dysplasia in a subject, the method comprising: analyzing the levels of amphiregulin in pancreatic cyst fluid samples derived from the subject before and after the subject undergoes said therapy, in conjunction with respective reference levels for amphiregulin. Increasing levels of amphiregulin in the subject indicate that the condition of the subject is worsening and decreasing levels of amphiregulin in the subject indicate that the condition of the subject is improving. The level of amphiregulin in pancreatic cyst fluid samples from the subject may be further compared to reference levels for amphiregulin for high grade dysplasia, cancer in situ, and invasive cancer.

In another embodiment, the invention includes a method for evaluating the effect of an agent for treating pancreatic cancer or dysplasia in a subject, the method comprising: analyzing the amount of amphiregulin in pancreatic cyst fluid samples derived from the subject before and after the subject is treated with the agent, and comparing the amount of amphiregulin with respective levels for amphiregulin.

In another aspect, the invention includes a biomarker panel comprising one or more biomarkers selected from the group consisting of glucose, kynurenine, and amphiregulin for diagnosis of pancreatic cysts. In one embodiment, the panel of biomarkers comprises glucose and kynurenine. In another embodiment, the panel of biomarkers comprises glucose, kynurenine, and amphiregulin.

In another aspect, the invention includes a kit for determining the diagnosis or prognosis of a subject having a pancreatic cyst. The kit may include one or more agents for detecting one or more biomarkers described herein, a container for holding a sample of pancreatic cyst fluid isolated from a subject; and printed instructions for reacting the agents with the sample of pancreatic cyst fluid or a portion of the sample to detect the presence or amount of one or more biomarkers in the sample of pancreatic cyst fluid. The agents may be packaged in separate containers. The kit may further comprise one or more control reference samples and reagents for performing a biochemical assay, enzymatic assay, immunoassay, or chromatography. In one embodiment, the kit may include an antibody that specifically binds to amphiregulin. In another embodiment, the kit may include reagents for performing a hexokinase-glucose-6-phosphate dehydrogenase coupled enzyme assay for detecting glucose. In another embodiment the kit may contain reagents for performing liquid chromatography (e.g., resin, solvent, and/or column).

These and other embodiments of the subject invention will readily occur to those of skill in the art in view of the disclosure herein.

### BRIEF DESCRIPTION OF THE FIGURES

FIG. 1 shows a scatter plot of cyst amphiregulin (AREG) by non-mucinous, benign mucinous, and malignant mucinous cysts.

FIG. 2 shows a receiver operator curve (ROC) analysis of AREG to differentiate benign mucinous from malignant mucinous cysts.

FIGS. 3A and 3B show a ROC analysis of glucose levels that differentiate mucinous from non-mucinous cysts. FIG. 3A shows that the area under the ROC for glucose to differentiate mucinous from nonmucinous cysts in the first cohort was 0.92 (95% CI 0.81-1.00). FIG. 3B shows that the area under the ROC for glucose to differentiate mucinous from non-mucinous cysts in the validation cohort was 0.88 (95% CI 0.72-1.00).

FIG. 4 shows scatterplots of cyst fluid glucose levels by non-mucinous and mucinous pancreatic cysts in two independent cohorts using the hexokinase-glucose-6-phosphate dehydrogenase spectrophotometric method. Mucinous cysts have significantly reduced glucose levels in both cohorts. The dashed line indicates the glucose level with the maximum empirical diagnostic performance (66 mg/dL) for distinguishing mucinous and non-mucinous cysts.

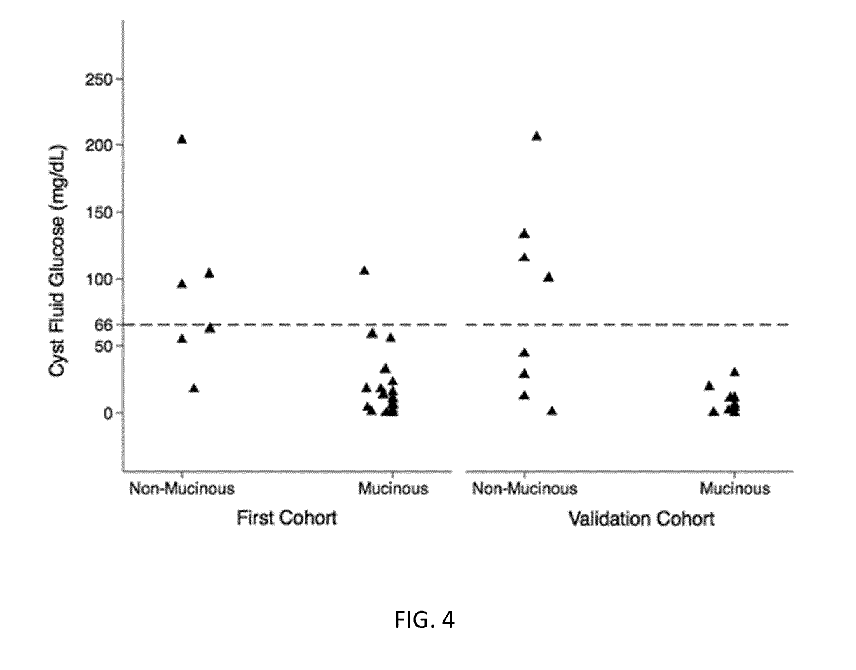

FIGS. 5A and 5B show a ROC analysis of kynurenine levels that differentiate mucinous from non-mucinous cysts. FIG. 5A shows that the area under the ROC for kynurenine to differentiate mucinous from nonmucinous cysts in the first cohort was 0.94 (95% CI 0.81-1.00). FIG. 5B shows that the area under the ROC for kynurenine to differentiate mucinous from non-mucinous cysts in the validation cohort was 0.92 (95% CI 0.76-1.00).

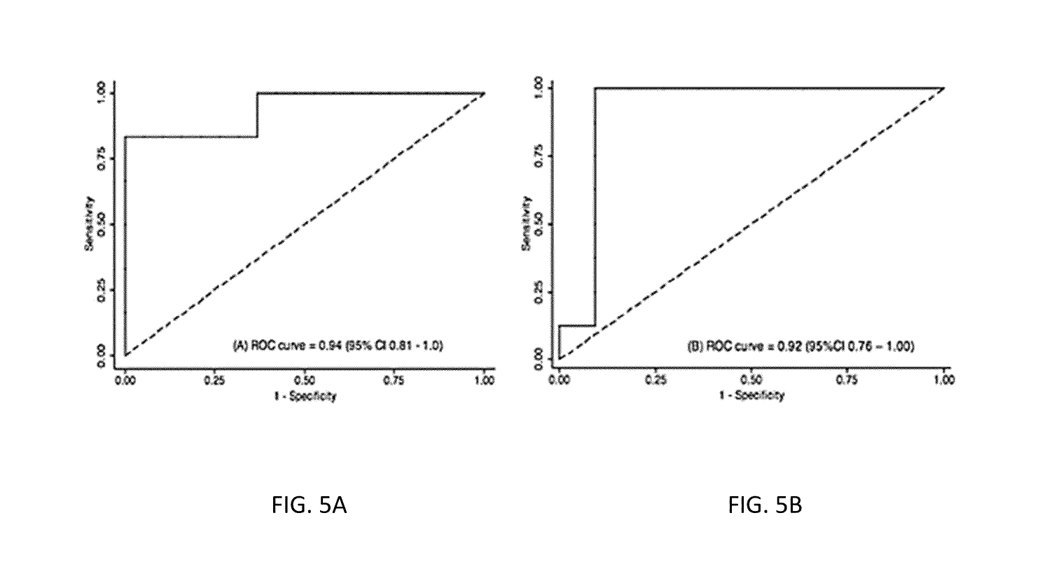

FIG. 6 shows a principle component analysis of metabolomic data. The x- and y-axis represent the 1^{st} and 2^{nd} principle components (PC), and the numbers in parenthesis indicate the proportion of the total variance explained by the corresponding PC. The shape indicates the type of cyst, and the color indicates whether it is in group 1 (non-mucinous, light gray) or group 2 (mucinous, dark gray). The pattern indicates that the metabolite levels can distinguish the mucinous and non-mucinous cysts.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Comprehensive Biomarker Discovery and Validation for Clinical Application (RSC Drug Discovery, P. Horvatovich, R. Bischoff, D. E. Thurston, D. Fox, D. Rotella, Royal Society of Chemistry, 2013); Jain The Handbook of Biomarkers (Humana Press, 2010 edition); Biomarkers: In Medicine, Drug Discovery, and Environmental Health (V. S. Vaidya and J. V. Bonventre eds., Wiley; 1^{st} edition, 2010); Pancreatic Cancer (J. P. Neoptolemos, R. A. Urrutia, J. Abbruzzese, M. W. Büchler eds., Springer; 2010 edition); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about”, particularly in reference to a given quantity, is meant to encompass deviations of plus or minus five percent.

A “biomarker” in the context of the present invention refers to a compound, such as a protein, a polypeptide or peptide fragment thereof, or a metabolite, which is differentially expressed in pancreatic cyst fluid of mucinous and nonmucinous cysts. Biomarkers include, but are not limited to, glucose, kynurenine, and amphiregulin.

“Metabolite” or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of metabolites include carbohydrates, amino acids, nucleotides, fatty acids, bile acids, steroids, hormones, lipids, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., LC-MS, GC-MS, NMR, biochemical or enzymatic assays, etc.), where the levels of biomarkers may differ based on the specific technique that is used.

A “similarity value” is a number that represents the degree of similarity between two things being compared. For example, a similarity value may be a number that indicates the overall similarity between a patient's expression profile using specific phenotype-related biomarkers and reference levels for the biomarkers in one or more control samples or a reference expression profile (e.g., the similarity to a mucinous pancreatic cyst expression profile or a non-mucinous pancreatic cyst profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The phrase “differentially expressed” refers to differences in the quantity and/or the frequency of a biomarker present in one sample compared to another, such as a pancreatic cyst fluid sample taken from a patient having, for example, a mucinous pancreatic cyst as compared to a non-mucinous pancreatic cyst or a sample of pancreatic cyst fluid taken from a benign pancreatic cyst compared to a malignant pancreatic cyst. For example, a biomarker can be a polypeptide or a metabolite, which is present at an elevated level or at a decreased level in pancreatic cyst fluid samples from patients with a particular type of pancreatic cyst compared to pancreatic cyst fluid samples from subjects with a different type of pancreatic cyst. Alternatively, a biomarker can be a polypeptide or metabolite which is detected at a higher frequency or at a lower frequency in pancreatic cyst fluid samples from patients with one type of pancreatic cyst compared to pancreatic cyst fluid samples from subjects with a different type of pancreatic cyst. A biomarker can be differentially present in terms of quantity, frequency or both.

A polypeptide or metabolite is differentially expressed between two samples if the amount of the polypeptide or metabolite in one sample is statistically significantly different from the amount of the polypeptide or metabolite in the other sample. For example, a polypeptide or metabolite is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide or metabolite is differentially expressed in two sets of samples if the frequency of detecting the polypeptide or metabolite in pancreatic cyst fluid samples from patients having a particular type of pancreatic cyst, is statistically significantly higher or lower than in pancreatic cyst fluid samples from patients having a different type of pancreatic cyst. For example, a polypeptide or metabolite is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a particular type of pancreatic cyst. A diagnostic amount can be either an absolute amount (e.g., μg/ml) or a relative amount (e.g., relative intensity of signals).

A “control amount” of a biomarker can be any amount or a range of amount which is to be compared against a test amount of a biomarker. For example, a control amount of a biomarker can be the amount of a biomarker in a non-mucinous pancreatic cyst. A control amount can be either an absolute amount (e.g., μg/ml) or a relative amount (e.g., relative intensity of signals).

A tissue has “malignant potential” if that tissue is likely to progress to cancer or already is cancerous. For example, a pancreatic cyst has malignant potential if that cyst is likely to develop into a mucinous cystic neoplasm or an intraductal papillary mucinous neoplasm.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as dansyl dyes, fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to metabolites, nucleic acid molecules, or proteins that are removed from their natural environment and are isolated or separated, and are at least about 60% free, preferably about 75% free, and most preferably about 90% free, from other components with which they are naturally associated.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of biomarkers in pancreatic cyst fluid that can be used in the diagnosis, prognosis, and treatment of pancreatic cysts. The inventors have shown that the levels of the protein, amphiregulin, in pancreatic cyst fluid can be used to distinguish between benign mucinous and malignant mucinous pancreatic cysts (see Example 1). In addition, the levels of certain metabolites in pancreatic cyst fluid, including glucose and kynurenine, can be used to distinguish mucinous and non-mucinous pancreatic cysts (see Example 2). Accordingly, these three biomarkers can be used in distinguishing different types of pancreatic cysts, including pseudocysts, serous cystadenomas, mucinous cystic neoplasms, and intraductal papillary mucinous neoplasms (see Examples 1 and 2). Based on identifying the type of pancreatic cyst present in a patient, a physician can recommend an appropriate course of action in treatment of the pancreatic cyst. For example, high levels of amphiregulin (greater than 300 pg/ml) in pancreatic cyst fluid from a subject indicate that a subject is at high risk of developing pancreatic cancer and in need of surgical removal of a malignant pancreatic cyst. On the contrary, high levels of glucose (greater than or equal to 66 mg/dL) or kynurenine in pancreatic cyst fluid indicate that a subject is at low risk of developing pancreatic cancer and that the subject has a benign, non-mucinous pancreatic cyst, which should remain under continued observation, but that does not require immediate surgical removal.

In order to further an understanding of the invention, a more detailed discussion is provided below regarding the identified biomarkers and methods of using them in prognosis, diagnosis, and monitoring treatment of pancreatic cysts.

A. Biomarkers

Biomarkers that can be used in the practice of the invention include, but are not limited to glucose, kynurenine, and amphiregulin. These biomarkers can be used alone or in combination with one or more additional biomarkers or relevant clinical parameters in prognosis, diagnosis, or monitoring treatment of pancreatic cysts. In certain embodiments, a panel of biomarkers comprising one or more biomarkers selected from the group consisting of glucose, kynurenine, and amphiregulin is used for prognosis, diagnosis, or monitoring treatment of pancreatic cysts. In one embodiment, the panel of biomarkers comprises glucose and kynurenine. In another embodiment, the panel of biomarkers comprises glucose, kynurenine, and amphiregulin. Expression profiles of glucose, kynurenine, and amphiregulin are useful for distinguishing different types of mucinous and non-mucinous pancreatic cysts and for assessing the risk of disease progression to pancreatic cancer.

In the methods of the invention, a sample of pancreatic cyst fluid is collected from a subject, and the levels of one or more biomarkers selected from the group consisting of glucose, kynurenine, and amphiregulin are measured and compared with reference levels for the biomarkers in mucinous and non-mucinous pancreatic cysts. The pancreatic cyst fluid sample obtained from the subject to be diagnosed is any fluid derived from a cystic lesion of the pancreas of the subject. The pancreatic cyst fluid sample can be obtained from the subject by conventional techniques well known in the art, such as endoscopic ultrasound (EUS) with fine needle aspiration or surgical collection. The reference levels for a biomarker can represent the amount of a biomarker found in one or more samples of one or more non-mucinous cysts. Alternatively, the reference levels for a biomarker can represent the amount of a biomarker found in one or more samples of one or more mucinous cysts. More specifically, the reference levels for a biomarker can represent the amount of a biomarker in a particular type of non-mucinous or mucinous pancreatic cyst (e.g., pseudocyst, serous cystadenoma, mucinous cystic neoplasm, or intraductal papillary mucinous neoplasm) to facilitate a determination of the type of pancreatic cyst present and the malignant potential of the pancreatic cyst in an individual.

For example, the level of glucose can be measured and compared to reference levels for glucose in pancreatic cyst fluid of mucinous and non-mucinous pancreatic cysts. A level of glucose greater than or equal to 66 mg/dL indicates that a pancreatic cyst is a non-mucinous pancreatic cyst. A level of glucose less than 66 mg/dL indicates that a pancreatic cyst is a mucinous pancreatic cyst.

In another example, the level of kynureinine can be measured and compared to reference levels for kynureinine in pancreatic cyst fluid of mucinous and non-mucinous pancreatic cysts. A lower level of kynurenine in a sample of pancreatic cyst fluid from a subject compared to the level of kynureinine in pancreatic cyst fluid from one or more benign non-mucinous cysts indicates that the pancreatic cyst in the subject is a mucinous pancreatic cyst.

In another example, the level of amphiregulin can be measured, wherein a level of amphiregulin greater than 300 pg/ml in pancreatic cyst fluid indicates that a pancreatic cyst is a malignant mucinous pancreatic cyst. Additionally, the level of amphiregulin in pancreatic cyst fluid samples from a subject may be compared to reference levels for amphiregulin for high grade dysplasia, cancer in situ, and invasive cancer to determine the stage of disease progression in an individual.

The measurement of biomarker levels in pancreatic cyst fluid has a number of applications. For example, biomarkers can be used to distinguish mucinous and non-mucinous cysts. The method comprises: a) obtaining a sample of pancreatic cyst fluid from a subject; b) measuring the levels of one or more biomarkers in the pancreatic cyst fluid, wherein the one or more biomarkers are selected from the group consisting of glucose and kynurenine; and c) analyzing the levels of one or more biomarkers in conjunction with respective reference levels for the biomarkers, wherein similarity of the levels of one or more biomarkers in the cyst fluid to reference value levels for a mucinous cyst indicates that the cyst in the subject is a mucinous cyst, and wherein similarity of the levels of one or more biomarkers in the cyst fluid to reference levels for a non-mucinous cyst indicates that the cyst in the subject is a non-mucinous cyst. In one embodiment, the method further comprises measuring the level of amphiregulin to distinguish malignant and non-malignant mucinous pancreatic cysts.

In another example, biomarkers can be used for monitoring a pancreatic cyst in a subject. The method comprises: a) analyzing a first pancreatic cyst fluid sample from a subject to determine the levels of one or more biomarkers, wherein the one or more biomarkers are selected from the group consisting of glucose, kynurenine, and amphiregulin, wherein the first sample is obtained from the subject at a first time point; b) analyzing a second pancreatic cyst fluid sample from the subject to determine the levels of the one or more biomarkers, wherein the second sample is obtained from the subject at a second time point; and c) comparing the levels of the one or more biomarkers in the first pancreatic cyst fluid sample to the levels of the one or more biomarkers in the second pancreatic cyst fluid sample in order to detect any changes in the status of the pancreatic cyst in the subject over time. For example, an initially benign cyst can be monitored over time and only surgically removed if changes in the levels of amphiregulin indicate that the cyst has undergone a transition to become a malignant cyst. If levels of glucose and kynureinine in the pancreatic cyst fluid indicate that the cyst is still benign, the cyst can remain under surveillance rather than be removed surgically.

In one embodiment, the invention includes a method for treating a pancreatic cyst in a subject, the method comprising: obtaining a sample of pancreatic cyst fluid from the pancreatic cyst in the subject, and surgically removing the pancreatic cyst from the subject if the level of amphiregulin in the pancreatic cyst fluid sample is greater than 300 pg/ml.

The methods of the invention, as described herein, can also be used for determining the prognosis of a subject and for monitoring treatment of a subject having pancreatic cysts. The inventors have shown that increased levels of amphiregulin in pancreatic cyst fluid are correlated with malignant mucinous pancreatic cysts and the likelihood of disease progression to pancreatic cancer (see, e.g., Example 1). Levels of amphiregulin above 300 pg/ml in pancreatic cyst fluid indicate that a subject has pancreatic cancer or high-grade dysplasia. Levels of glucose greater than or equal to 66 mg/dL in pancreatic cyst fluid indicate that the pancreatic cyst is a benign non-mucinous pancreatic cyst and that the subject has a low risk of disease progression to pancreatic cancer.

Thus, a medical practitioner can monitor the progress of disease by measuring the level of the biomarkers in pancreatic cyst fluid samples from the patient. For example, a decrease in the level of amphiregulin or an increase in the level of glucose or kynurenine as compared to a prior level of amphiregulin, glucose or kynurenine (e.g., in a prior pancreatic cyst fluid sample) indicates the disease or condition in the subject is improving or has improved, while an increase of the amphiregulin level or decrease in the level of glucose or kynurenine as compared to a prior level of amphiregulin, glucose or kynurenine (e.g., in a prior sample of pancreatic cyst fluid) indicates the disease or condition in the subject has worsened or is worsening. Such worsening could possibly result in the subject developing pancreatic cancer or high grade dysplasia.

The methods described herein for prognosis or diagnosis of subjects having pancreatic cysts, who are at risk of having pancreatic cancer or dysplasia, may be used in individuals who have not yet been diagnosed (for example, preventative screening), or who have been diagnosed, or who are suspected of having pancreatic cancer or dysplasia (e.g., display one or more characteristic symptoms), or who are at risk of developing pancreatic cancer or dysplasia (e.g., have a genetic predisposition or presence of one or more developmental, environmental, or behavioral risk factors). The methods may also be used to detect various stages of progression or severity of disease. The methods may also be used to detect the response of disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

In one embodiment, the invention includes a method for monitoring the efficacy of a therapy for treating pancreatic cancer or dysplasia in a subject. The method comprises: analyzing the levels of amphiregulin in pancreatic cyst fluid samples derived from the subject before and after the subject undergoes said therapy, in conjunction with respective reference levels for amphiregulin. Increasing levels of amphiregulin in the subject indicate that the condition of the subject is worsening and decreasing levels of amphiregulin in the subject indicate that the condition of the subject is improving. The level of amphiregulin in pancreatic cyst fluid samples from the subject may be further compared to reference levels for amphiregulin for high grade dysplasia, cancer in situ, and invasive cancer to determine the stage of disease progression.

In another embodiment, the invention includes a method for evaluating the effect of an agent for treating pancreatic cancer or dysplasia in a subject. The method comprising: analyzing the levels of amphiregulin in pancreatic cyst fluid samples derived from the subject before and after the subject is treated with the agent, and comparing the amount of amphiregulin with respective reference levels for amphiregulin.

B. Detecting and Measuring Levels of Biomarkers

It is understood that the expression levels of the biomarkers in a sample of pancreatic cyst fluid can be determined by any suitable method known in the art. Suitable methods include chromatography (e.g., high-performance liquid chromatography (HPLC), gas chromatography (GC), liquid chromatography (LC)), mass spectrometry (e.g., MS, MS-MS), NMR, enzymatic or biochemical reactions, immunoassay, and combinations thereof. Measurement of the expression level of a biomarker can be direct or indirect. For example, the abundance levels of proteins or metabolites can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker.

The metabolite biomarkers, glucose and kynurenine, can be measured, for example, by mass spectrometry or NMR using metabolomic profiling techniques well known in the art. Mass spectrometry can be combined with chromatographic methods, such as liquid chromatography (LC), gas chromatography (GC), or electrophoresis to separate the metabolite being measured from other components in the pancreatic cyst fluid. See, e.g., Hyötyläinen (2012) Expert Rev. Mol. Diagn. 12(5):527-538; Beckonert et al. (2007) Nat. Protoc. 2(11):2692-2703; O'Connell (2012) Bioanalysis 4(4):431-451; and Eckhart et al. (2012) Clin. Transl. Sci. 5(3):285-288; herein incorporated by reference. Alternatively, metabolites can be measured with biochemical or enzymatic assays (see, e.g., Example 2). For example, glucose can be measured with a hexokinase-glucose-6-phosphate dehydrogenase coupled enzyme assay. In another example, biomarkers can be separated by chromatography and relative levels of a biomarker can be determined from analysis of a chromatogram by integration of the peak area for the eluted biomarker.

Immunoassays based on the use of antibodies that specifically recognize a biomarker may be used for measurement of biomarker levels. Such assays include, but are not limited to, enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF), the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 3142, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is pancreatic cyst fluid, a protein microchip (e.g., See Arenkov P, et al., Anal Biochem., 278(2):123-131 (2000)), or a chromatography column with bound biomarkers, etc.; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

It may be useful in the practice of the invention to fractionate pancreatic cyst fluid samples, e.g., to enrich samples for lower abundance biomarkers to facilitate detection of biomarkers. There are many ways to reduce the complexity of a sample based on the properties of the biomarkers in the sample.

In one embodiment, a sample can be fractionated according to the size of the biomarker in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the biomarkers in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain biomarkers, and an eluant containing non-adsorbent biomarkers (i.e., biomarkers that did not bind to the first adsorbent) is collected. Then, the fraction is exposed to a second adsorbent. This further extracts various biomarkers from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution, facilitate detection, or to determine its identity. For example, protein biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is useful for detection of biomarkers. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting biomarkers. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as proteins or metabolites, in complex mixtures. SELDI is a method of mass spectrometry in which biomolecules, such as proteins or metabolites, are captured on the surface of a biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, the biomarkers are useful in distinguishing benign and malignant pancreatic cysts and can be used in the diagnosis or prognosis of pancreatic cysts. In another example, the biomarkers can be used to identify patients with a high risk of progression to pancreatic cancer, who are in need of surgical removal of a malignant pancreatic cyst. In another example, the methods for detection of the biomarkers can be used to monitor responses in a subject to treatment. In yet another example, the methods for detecting biomarkers can be used to assay for and to identify compounds that modulate expression of these biomarkers in vivo or in vitro.

C. Kits

In yet another aspect, the invention provides kits for diagnosis or prognosis of a subject having a pancreatic cyst, wherein the kits can be used to detect at least one biomarker selected from the group consisting of glucose, kynurenine, and amphiregulin. For example, the kits can be used to detect any one or more of the biomarkers described herein, which are differentially expressed in samples of pancreatic cyst fluid from mucinous and non-mucinous pancreatic cysts or benign and malignant pancreatic cysts. The kit may include one or more agents for detection of one or more biomarkers, a container for holding a sample of pancreatic cyst fluid isolated from a subject; and printed instructions for reacting agents with the sample of pancreatic cyst fluid or a portion of the sample to detect the presence or amount of one or more biomarkers in the sample. The agents may be packaged in separate containers. The kit may further comprise one or more control reference samples and reagents for performing a biochemical assay, enzymatic assay, immunoassay, or chromatography. In one embodiment, the kit may include an antibody that specifically binds to amphiregulin. In another embodiment, the kit may include reagents for performing a hexokinase-glucose-6-phosphate dehydrogenase coupled enzyme assay for detecting glucose. In another embodiment the kit may contain reagents for performing liquid chromatography (e.g., resin, solvent, and/or column)

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of distinguishing different types of mucinous and non-mucinous pancreatic cysts (e.g., pseudocyst, serous cystadenoma, mucinous cystic neoplasm, or intraductal papillary mucinous neoplasm).

The kits of the invention have a number of applications. For example, the kits can be used to determine if a subject has a benign or malignant pancreatic cyst. In another example, the kits can be used to determine the likelihood of disease progression to pancreatic cancer for a subject having a pancreatic cyst and the need for surgical intervention. In another example, kits can be used to monitor the effectiveness of a treatment of a patient having a pancreatic cyst. In a further example, the kits can be used to identify compounds that modulate expression of the biomarkers in in vitro or in vivo animal models to determine the effects of treatment.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Diagnostic Accuracy of Cyst Fluid Amphiregulin in Pancreatic Cysts

In this study, the diagnostic utility of the secreted epidermal growth factor receptor ligand, amphiregulin (AREG), was explored as a cyst fluid biomarker for the presence of malignancy in pancreatic cysts. AREG was chosen based on previous gene expression studies that identified enhanced Anterior Gradient 2 (AGR2) expression in all pancreatic adenocarcinomas (Lowe et al. (2007) PLoS One 2:e323). AGR2 stimulates adenocarcinoma cell growth and supports the development of many features associated with malignant transformation (Wang et al. (2008) Cancer Res. 68(2):492-497; Ramachandran et al. (2008) Cancer Res. 68(19):7811-7818). A recent study demonstrated that AGR2's growth promoting properties are achieved through its induction of AREG expression in adenocarcinoma cells (Dong et al. (2011) J. Biol. Chem. 286(20):18301-18310). As a secreted molecule, we hypothesized that the AREG concentration within the cyst fluid of adenocarcinomas or high-grade dysplastic lesions possesses diagnostic utility in the evaluation of pancreatic cysts.

Methods

Cyst Fluid Samples

With the approval of the Stanford University Human Subjects Institutional Review Board, a pancreatic cyst fluid bio-repository has been maintained since July 2008. Patients evaluated at Stanford Hospital and Clinics for endoscopic ultrasound or surgery for pancreatic cysts were offered participation in the study. Cyst fluid was collected at the time of endoscopic ultrasound and/or surgery. Patients with a cyst large enough (typically greater than 1 cm) to provide cyst fluid beyond what was required for clinical evaluation was immediately placed on ice, aliquoted, and stored at −80° C. Clinical evaluation of the cyst fluid primarily involved 500 microliters of fluid for carcinoembryonic antigen (CEA) analysis. Testing for amylase was left to the clinical discretion of the gastroenterologist or surgeon. When an intracystic nodule was seen, the nodule underwent fine needle aspiration for tissue diagnosis. All samples were aliquoted and frozen at −80° C. within 30 minutes of collection. All samples assayed were subjected to no more than two freeze-thaw cycles, which does not affect the assay's reproducibility.

Diagnosis of Pancreatic Cysts

Cyst diagnosis was determined by surgical pathology or cytology. In each of the surgically resected cases, histology slides were independently evaluated by a pathologist (RKP) for the histology type and grade of the neoplasm. All cases of intraductal papillary mucinous neoplasms (IPMN) and mucinous cystic neoplasms (MCN) were subclassified based on the grade of dysplasia: low-grade, intermediate-grade, and high-grade, using the WHO classification (Cancer TIAfRo: WHO Classification of Tumors of the Digestive System (IARC WHO Classification of Tumors), Edited by: Bosman F. T., Carneiro, G., Hruban, R. H., Theise, N. D. World Health Organization; 4 2010). In this study, the definition of cancer included cystic lesions with high-grade dysplasia. Benign mucinous cysts included MCN or IPMN lesions with low- or intermediate-grade dysplasia.

AREG ELISA

Researchers (M.T.T., A.W.L.) blinded to the patients' diagnoses conducted the AREG ELISAs. Cyst fluid AREG was determined using a two-antibody sandwich ELISA (DY262, R&D systems, Minneapolis, Minn.) according to the manufacturer's instructions. Standard curves were reproducible over a dynamic range of 5-2,000 pg/ml. Briefly, 100 microliter (μl) of sample was required for analysis and added to a 96-well ELISA plate (Fisher Scientific, Pittsburg, Pa.) that had been pre-coated with the capture antibody. After incubation with the detection antibody and streptavidin-HRP, the signal was developed by the addition of 3,3′,5,5′-tetramethyl-benzidine (TMB, Thermo Scientific, Rockford, Ill.), followed by the addition of a stop solution, and quantified by absorptive spectrophotometry at 450 and 562 nm on an automatic plate reader (Biotek, Winooski, Vt.). Assays for each sample were performed on serially diluted aliquots and performed in duplicate. The diluent consisted of 1% bovine serum albumin in phosphate buffered saline, pH 7.3. Dilutions within the assay's linear range on the standard curve were chosen. Data demonstrating that the ELISA specifically measures the AREG gene product was previously established (Dong et al. (2011) J. Biol. Chem. 286(20):18301-18310).

Statistical Analysis

Comparisons between mucinous and non-mucinous cysts and benign mucinous and malignant mucinous cysts were performed. Based on a non-normal distribution of AREG levels by cyst type, the non-parametric Kruskal-Wallis test was used to compare AREG levels between the multiple categories of cysts. The Wilcoxon rank-sum test was used for comparison of 2 cyst types. A receiver operator curve was generated to characterize the accuracy of cyst fluid AREG to diagnose malignant mucinous cysts. When a significant difference was observed, a threshold with highest diagnostic accuracy was used to report the sensitivity and specificity of AREG. Statistical analysis was performed using STATA 11.0 (College Station, Tex.).

Results

Patients and Cyst Types

Thirty-three patients with pancreatic cysts were evaluated (Table 1). The mean age was 61 (range 33-83) and 54% (18 of 33) were males. The median cyst size was 2.8 cm (interquartile range [IQR] 2.0-4.4 cm). A histological diagnosis was conferred by surgical pathology for 30 samples and by cyst aspiration cytology for 3 samples. Among the 30 surgical pathology samples, there were 5 adenocarcinomas, 4 cysts with high-grade dysplasia (all MD-IPMN), 15 benign mucinous cysts (MCN=3, BD-IPMN=9, and MD-IPMN=3), and 6 non-mucinous cysts (SCN=4, PC=1, squamous cyst=1). Histological samples conferred only by cytology (n=3) were cysts associated with unresectable adenocarcinoma.

Diagnostic Accuracy of AREG

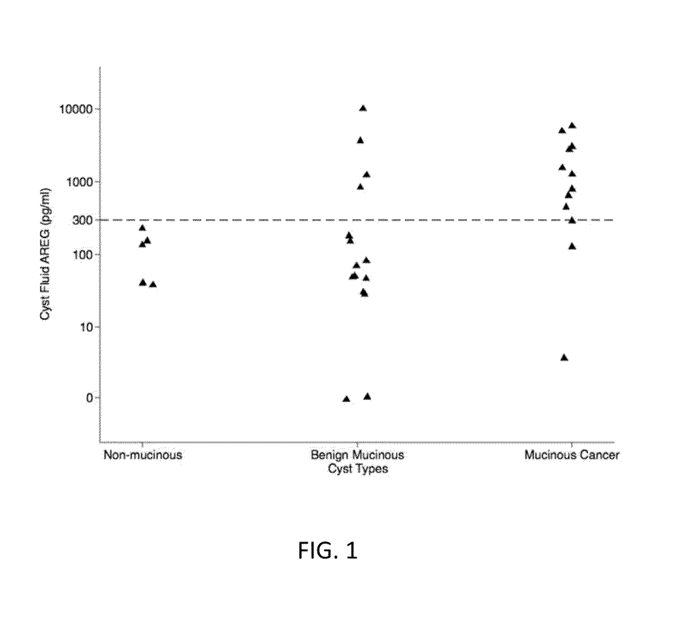

Scatter plots of cyst AREG levels by cyst type are shown in FIG. 1. The median (interquartile range, IQR) cyst AREG levels for non-mucinous cysts, benign mucinous cysts, and cancerous cysts were 85 pg/ml (47-168), 63 pg/ml (30-847), and 986 pg/ml (417-3160), respectively. Table 2 summarizes cyst AREG values by each type of cyst. No significant difference in AREG levels was appreciated between non-mucinous and mucinous cysts. When mucinous cysts were divided between benign and cancerous cysts a significant difference in cyst AREG levels was observed (p=0.025).

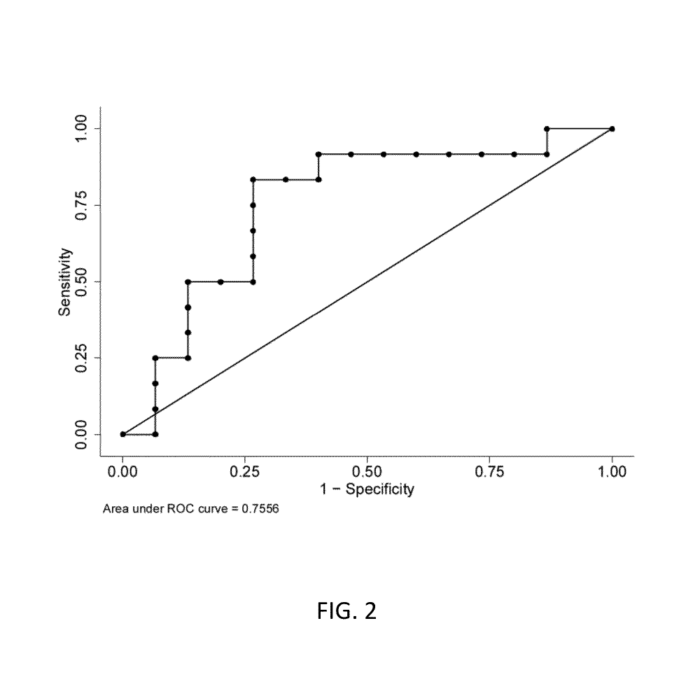

Based on the difference of cyst AREG levels between benign mucinous and mucinous cancers, a receiver operator curve (ROC) was generated to determine an optimal threshold to diagnose mucinous cancers (FIG. 2). As a summary measure of diagnostic accuracy, the area under the ROC was 0.76 (95% CI 0.56-0.95). At an AREG threshold of greater than 300 pg/ml, the diagnostic accuracy for cancer was 78% with a sensitivity of 83% and specificity of 73%. With the prevalence of cancer of 32% in the sample, the positive and negative predictive value was 71% and 85%, respectively.

Further clinical details on the 12 patients with cancer in this sample are highlighted in Table 3. Four patients had high-grade dysplastic lesions, and included 3 MDIPMN. The majority of patients (10 out of 12) had symptoms (i.e. jaundice, weight loss, abdominal pain) associated with cancer. The majority of patients had imaging evidence of a nodule within the cyst or an associated mass (8 out of 12). Two out of the 12 cases had AREG levels below 300 pg/ml. One case (AREG=125) was an intraductal oncocytic papillary neoplasm and the other case (AREG=4) was a 1.5 cm cyst adjacent to a pancreatic adenocarcinoma.

TABLE 1
Summary of Patient and Cyst Characteristics

| Total Patients | 33 | |
|---|---|---|
| Median Age, years (range) | 61 | (33-83) |
| Gender: Male/Female | 18 (54%)/15 (46%) | |
| Median Cyst Size, cm (IQR) | 2.8 | (2.0-4.4) |

| | |
|---|---|
| Non-Mucinous | 6 |
| SCN (n = 4) | |
| Pseudocyst (n = 1) | |
| Other (n = 1) | |
| Benign Mucinous | 15 |
| IPMN BD (n = 9) | |
| IPMN MD (n = 3) | |
| MCN (n = 3) | |
| Cancer (in situ) | 12 |
| High Grade (n = 4) | |
| Invasive (n = 8) | |

TABLE 2
Summary of Cyst Fluid AREG performance by Cyst Types

| | Median AREG | |
|---|---|---|
| Cyst Type (n = 33) | (pg/ml) | IQR (pg/ml) |
| Non-Mucinous (n = 6) | 85 | 47-168 |
| SCN (n = 4) | 48 | 44-109 |
| Pseudocyst (n = 1) | 227 | |
| Other (n = 1) | 121 | |
| Benign Mucinous (n = 15) | 63 | 30-847 |
| IPMN BD (n = 9) | 48 | 29-63 |
| IPMN MD (n = 3) | 847 | 71-9041 |
| MCN (n = 3) | 202 | 42-1030 |
| Cancer (in situ) | 986 | 417-3160 |
| High Grade (n = 4) | 417 | 214-546 |
| Invasive (n = 8) | 2047 | 986-4367 |

TABLE 3
Summary Table of 12 patients with histological diagnosis
of Cancer (includes high grade dysplasia)

| Patient | | Cyst | | Mural | AREG | CEA | |
|---|---|---|---|---|---|---|---|
| Age/ | | Size | | Nodule/ | level | level | |
| Gender | Symptomatic | (cm) | Location | Mass | (pg/ml) | (ng/ml) | Diagnosis |
| 39/F | Yes | 2.8 | Tail | Yes | 125 | 15 | Intraductal Oncocytic |
| | | | | | | | Papillary Neoplasm |
| 72/M | No | 4.6 | Body | No | 303 | 2298 | Main Duct IPMN with |
| | | | | | | | High-Grade Dysplasia |
| 78/M | Yes | N/A | Diffuse | No | 523 | N/A | Main Duct IPMN with |
| | | | | | | | High-Grade Dysplasia |
| 65/M | Yes | N/A | Diffuse | No | 560 | N/A | Main Duct IPMN with |
| | | | | | | | High-Grade Dysplasia |
| 60/M | No | 1.5 | Body | Yes | 4 | 1245 | Adenocarcinoma |
| 83/M | Yes | 3.0 | Body | Yes | 694 | 42979 | Adenocarcinoma |
| 60/M | Yes | 3.0 | Tail | Yes | 1279 | N/A | Adenocarcinoma |
| 60/F | Yes | 3.2 | Tail | Yes | 1567 | 11962 | Adenocarcinoma |
| 66/F | Yes | 2.6 | Head | Yes | 2527 | N/A | Adenocarcinoma |
| 70/M | Yes | N/A | Diffuse | No | 3794 | N/A | Colloid Carcinoma |
| 51/M | Yes | 3.0 | Head | Yes | 4940 | N/A | Adenocarcinoma |
| 65/M | Yes | 7.5 | Head | Yes | 6458 | 2501 | Adenocarcinoma |

Discussion

A biomarker that can accurately and reliably distinguish cancer or high-grade dysplasia among mucinous pancreatic cystic neoplasms remains an important clinical need. The most accepted cyst fluid biomarker currently is CEA, which is good at differentiating mucinous from non-mucinous cysts. CEA, however, is not reliable for differentiating cancer or high-grade dysplasia among pre-malignant mucinous cysts. As a result, current practice relies on clinical and radiographic data to help clinicians decide which cystic lesions warrant immediate surgery over observation (Tanaka et al. (2006) Pancreatology, 6(1-2):17-32). While helpful, cases of unnecessary surgery or missed opportunities to resect cancer occur (Pelaez-Luna et al. (2007) Am. J. Gastroenterol. 102(8):1759-1764; Correa-Gallego et al. (2010) Pancreatology 10(2-3):144-150; Walsh et al. (2008) Surgery 144(4):677-684, discussion 84-85).

AREG's discovery as a potential cyst fluid biomarker arose from observations of increased Anterior Gradient 2 (AGR2) gene expression among pancreatic adenocarcinomas (Lowe et al. (2007) PLoS One 2:e323). AGR2 is a highly conserved gene that is associated with mucus secreting cells. AGR2 stimulates adenocarcinoma cell growth and supports the development of many features associated with malignant transformation (Wang et al. (2008) Cancer Res. 68(2):492-497; Ramachandran et al. (2008) Cancer Res. 68(19):7811-7818). Closer examination of the gene expression studies showed that AGR2 expression was significantly higher in MCN cysts compared to SCA lesions. Recent studies revealed that AREG, a secreted epidermal growth factor receptor ligand, is specifically induced by AGR2 (Dong et al. (2011) J. Biol. Chem. 286(20):18301-18310).

In this study, we examined the diagnostic utility of AREG in pancreatic cyst fluid and observed no difference in cyst AREG concentrations between non-mucinous and benign mucinous cysts. Malignant mucinous cysts that included high-grade dysplastic lesions, however, expressed a significantly higher AREG level (median 986 pg/ml) compared to benign mucinous cysts (median 63 pg/ml) and non-mucinous cysts (median 85 pg/ml). By receiver operator curve analysis, an AREG level of 300 pg/ml provided a diagnostic accuracy for cancer of 78% (sensitivity 83%, specificity 73%). The higher cyst AREG levels observed in malignant cysts is likely a function of the total cellular mass of AREG producing cells. As a benign cyst transitions to a malignant cyst, a hallmark of dysplasia includes a change from simple to stratified epithelium. We hypothesized that this results in a significant increase in the cellular mass of a cyst leading to increased cyst AREG expression. The similarities in cyst AREG levels between non-mucinous and benign mucinous cysts may be related to the physiologic expression of AREG as part of a reparative process in combination with a smaller cellular mass of mucin producing cells. Recent studies have determined that AREG serves an important role in tissue repair after damage in the gastrointestinal tract (Shao et al. (2010) Endocrinology 151(8):3728-3737; Berasain et al. (2005) Gastroenterology 128(2):424-432).

Because cyst CEA is fairly accurate in differentiating non-mucinous from mucinous cysts, the diagnostic utility of combining both CEA and AREG was considered. There were 21 of the 33 samples where cyst fluid CEA and AREG levels were available for analysis. The median (IQR) CEA levels for the 4 non-mucinous cysts, 11 benign mucinous, and 6 malignant mucinous cysts were 127 ng/ml (36-844), 1294 ng/ml (171-8600), and 2400 ng/ml (1245-11962), respectively. Mucinous cysts (n=17) had an elevated CEA (median (IQR) 1311 ng/ml (277-8600)) compared to non-mucinous cysts (n=4) (126 ng/ml (36-844) (p=0.09). Although this difference was not statistically significant, this is likely due to the small sample size. Using a cutoff of 192 ng/ml, the sensitivity and specificity of CEA to differentiate non-mucinous from mucinous cysts was 76% and 75%, respectively—an observation similar to previous reports (Brugge et al. (2004) Gastroenterology 126(5):1330-1336; Park et al. (2011) Pancreas 40(1):42-45). The small size of this sample may also explain why no difference in sensitivity and specificity for cancer was observed when combining AREG and CEA compared to AREG alone. When an AREG threshold of 300 pg/ml was used for the diagnosis of malignant mucinous cysts, the sensitivity was 67% and the specificity 80%. When AREG was sequentially tested only on pancreatic cysts with a CEA level greater than 192 ng/ml, neither the sensitivity nor specificity changed for cancer.

There are several features of this study that limit the generalizability of these observed results. First, this is a retrospective single tertiary center with a relatively small sample of cyst fluid samples. The small sample size is due in part to restricting the study to surgical patients. Although recruitment was difficult because patients with pancreatic cysts often do not undergo surgery, it was felt that as an initial proof-of-concept study, the use of pathology and surgically resected samples was a necessary gold standard to establish the correct diagnosis. As a result, the impact of a small sample size (in particular the limited cases of non-mucinous cysts) may include inadequate power to demonstrate a difference between non-mucinous and mucinous AREG levels should one truly exist. Second, the 12 cancer cases (including high grade dysplasia) were relatively advanced cases and could likely be identified by current practices without cyst AREG. It is unclear how AREG will perform in cases when imaging and clinical characteristics are non-specific. Many of these limitations, however, can be addressed in the future with prospective, longitudinal validation incorporating a larger sample size and multi-center collaboration.

Conclusions

The present study represents the translation of recent discoveries in the basic biology of adenocarcinomas to clinical utility in the evaluation of pancreatic cysts. The study reports the discovery of AREG, a secreted epidermal growth factor receptor ligand, as a biomarker with potential diagnostic utility for diagnosing and managing pancreatic cystic neoplasms. Specifically, cyst AREG levels may help accurately identify those cysts with cancer and high-grade dysplastic lesions that require immediate surgical attention. Although not a serum-based test, EUS mediated acquisition of the 100 microliters of fluid necessary for analysis is within current practices for managing pancreatic cysts, and will facilitate validation in future studies.

### Example 2

### Metabolomic-Derived Novel Cyst Fluid Biomarkers for Pancreatic Cysts: Glucose and Kynurenine

To identify novel cyst fluid biomarkers, we used a metabolomic approach to identify uniquely expressed metabolites in clinically relevant pancreatic cyst types. Within the “-omics” cascade of discovering differences among different disease states, genomics focuses on what can happen, proteomics focuses on what makes it happen, and metabolomics focuses on what has happened and is happening (Dettmer et al. (2007) Mass Spectrom. Rev. 26(1):51-78). Metabolomic analysis can reveal a great deal about the physiological state of a tissue. However, the extreme differences in physicochemical properties make it impossible to accurately measure changes in all metabolites with a single analytic method.

In this study, we used a recently developed Dansyl [5-(dimethylamino)-1-napthalene sulfonamide] derivatization method (Guo et al. (2009) Anal. Chem. 81(10):3919-3932) and liquid chromatography with mass spectrometry (LC/MS) analysis to robustly analyze changes in many metabolites in pancreatic cyst fluid aspirates. Dansylation increases metabolite detection sensitivity by 10-1000 fold and improves metabolite identification. It enables changes in many metabolites to be evaluated in an unbiased fashion. This semi-targeted method was used to profile the metabolites in pancreatic cyst fluid obtained from two cohorts of individuals with pancreatic cysts defined by histology.

### Methods

Pancreatic Cyst Fluid Collection and Clinical Cohorts

An IRB-approved biorepository for pancreatic cyst fluid has been maintained at the Stanford University Medical Center since July 2008. Cyst fluid samples were obtained from patients with pancreatic cysts that were evaluated at Stanford Hospital and Clinics by endoscopic ultrasound or surgery. All procedures and sample collections were performed after informed consent was obtained according to an IRB-approved protocol. The cyst fluid that was obtained during endoscopic ultrasound (EUS) and/or surgical procedures and not needed for clinical care was immediately placed on ice, divided into aliquots, and stored at −80° C. All samples were frozen within 30 minutes of collection. No samples underwent more than 2 freeze-thaw cycles prior to evaluation. All included cysts were defined by histology from surgery (n=40) or positive cytology (n=5). We defined cancer to include high-grade dysplasia, pancreatic adenocarcinomas with cystic degeneration, and intraductal papillary mucinous neoplasm (IPMN)-associated cancers.

The first (derivation) cohort was developed by choosing consecutive samples with available histology of each cyst type with a goal of making the sample as balanced as possible of different cyst types. Since the clinical goal is to not operate on benign cysts like serous cystadenomas (SCA) and pseudocysts (PC), it was difficult to achieve an equal number of non-mucinous cysts to mucinous cysts. The validation cohort was developed after the derivation cohort using the same consecutive selection method.

Metabolomic Analysis

Four volumes of acetonitrile:methanol:Acetone (1:1:1 by volume) were added to one volume (50 μl) of pancreatic cyst fluid, then incubated at −20° C. for one hour. Dansylation was performed using a modification of the procedures developed by Guo and Li (Anal Chem. (2009) 81(10):3919-3932; herein incorporated by reference). A half volume of 0.1M sodium tetraborate buffer was added to one volume of the metabolite extract, and then combined with one volume of 50 mM dansyl chloride and vortexed. The mixture was incubated at room temperature for 30 minutes before addition of one volume of 0.5% formic acid to stop the reaction. The supernatant of the reaction mixture was then placed into an autosampler vial. All samples were then analyzed on an Agilent (Santa Clara, Calif.) accurate mass Q-TOF 6520 coupled with an Agilent UHPLC infinity 1290 system. The chromatography runs were performed using a Phenomenex (Torrance, Calif.) Kinetex reversed phase C18 column (dimension 2.1×100 mm, 2.6 mm particles, 100 Å pore size). Solvent A was HPLC water with 0.1% formic acid and Solvent B was LC/MS grade acetonitrile with 0.1% formic acid. A 30 minute gradient at 0.5 ml/min was as follows: t=0.5 minute, 5% B; t=20.5 minutes, 60% B; t=25 minutes, 95% B; t=30 minute, 95% B. The column was balanced at 5% B for 5 minutes. All data were acquired by positive ESI (electrospray ionization) with Masshunter acquisition software. Molecular feature extraction on all data was performed using Masshunter qual software. The metabolite abundance, which is a measure of the metabolite concentration in an extract, was determined by integration of the peak area for the indicated metabolite on the extracted ion chromatogram for each sample.

Glucose Assay

Based on metabolite abundance results, cyst fluid glucose levels were measured using an adaptation of the hexokinase-glucose-6-phosphate dehydrogenase spectrophotometric method, which was performed on a Dimension RxL analyzer (Siemens Healthcare Diagnostics, Deerfield, Ill.). See Kunst A, Draeger B, Zeigenhorn J. UV methods with exokinase and glucose-6-phosphate dehydrogenase. In: Bergmeyer H U, editor. Methods of Enzymatic Analysis. 6. Deerfield, F L: Verlag Chemie; 1983. p. 163-172; herein incorporated by reference. The reportable range for this assay is 5-500 mg/dL, with an intra- (n=20) and inter-assay (n=20) coefficient of variation of 0.6% and 1.2% at 88 mg/dL and 0.3% and 1.3% at 276 mg/dL, respectively. To minimize metabolism, all samples were processed within 15 minutes after complete thawing. Each sample (50 μL) was measured twice and the average was used for analysis. When the measured glucose value in the cyst fluid glucose was below 5 mg/dL, the actual value is less precisely determined, and a 5 mg/dL value was used in these analyses.

Statistical Analyses

The Kruskal-Wallis and Wilcoxon rank-sum tests were used to evaluate quantitative differences in cyst fluid glucose and kynurenine among cyst types and between non-mucinous and mucinous cysts, and mucinous non-cancerous cysts and cancerous cysts. The Chi square test was used for comparing proportions between the two cohorts when appropriate. A two-sample t-test with unequal variance was used on the log-scale of the data to compare the abundance measured by Mass Spectrometry. To compare the diagnostic accuracy of combining 2 biomarkers to each alone, a conditional binomial test was applied. Statistical analysis was performed using STATA 11.0 (College Station, Tex.).

Principle component analysis (PCA) was used to investigate the pattern of metabolite changes in an unbiased and unsupervised manner. For this analysis, the metabolomic data in the 5 different cysts categories (SCA, PC, mucinous cystic neoplasms (MCN), IPMN, and cancer) was obtained, and all metabolites that were present in more than one sample were included. The minimum threshold abundance was empirically set to 1000. If a metabolite was undetected in a sample (i.e. the abundance was less than the threshold), it was then assumed that its true abundance was between 0 and 1000; and metabolite abundance in that sample was assumed to be half of the threshold value. The data then underwent a log 10-based-transformation, and PCA (Hastie T, Tibshirani R, Friedman J. The Elements of Statistical Learning. New York: Springer; 2001) was used to display the data. The PCA was performed in R (r-project.org).

### Results

Clinical Cohorts

Clinical and relevant imaging characteristics of 2 independent clinical cohorts for metabolite analysis are displayed in Table 1. There were no significant differences in age, gender distribution, or cyst size between these two cohorts. The mean age was 62 years in the first cohort and 59 years in the validation cohort with a slight predominance of males. The median cyst size was 3.0 cm in the first cohort and 3.2 cm in the validation cohort. As a reason for surgical resection, 62% of patients in the first cohort had one of the following high-risk features: main duct dilation, solid component, or associated symptoms. Associated symptoms included abnormal weight loss, jaundice, or acute pancreatitis. In the validation cohort, 58% of patient had one of the following high-risk features. There was no significant difference in high-risk features between the 2 cohorts (p=0.8).

TABLE 1
Clinical Characteristics of Cohorts

| | First | | Validation | | |
|---|---|---|---|---|---|
| | Cohort | | Cohort | | p-value |
| Total Patients | 26 | | 19 | | |
| Mean Age, years | 62 | (33-83) | 59 | (30-78) | 0.49 |
| (Range) | | | | | |
| Gender: Male/Female | 14 (54%)/12 (46%) | | 11 (58%)/8 (42%) | | 0.78 |
| Median Cyst Size, | 3.2 | (2.0-6.1) | 3.0 | (2.0-5.4) | 0.69 |
| cm (IQR) | | | | | |

| | | | |
|---|---|---|---|
| High-Risk Features | 62% | 58% | 0.8 |
| Main Duct Dilation | 15% | 26% | 0.36 |
| (%) | | | |
| Solid Component (%) | 31% | 16% | 0.24 |
| Associated | 42% | 37% | 0.71 |
| Symptoms (%)* | | | |

*Weight loss, Jaundice, Pancreatitis

Table 2 displays the type and frequency of pancreatic cysts in each cohort. The first cohort of 26 individuals included 6 non-mucinous (4 SCA and 2 PC) and 20 mucinous (4 MCN, 6 IPMN, and 10 cancer) cysts. The validation cohort of 19 individuals included 8 non-mucinous (4 SCA and 4 PC) and 11 mucinous (1 MCN, 8 IPMN, and 2 cancer) cysts.

TABLE 2
The median and inter-quartile (IQR) cyst glucose levels (measured using
a standard hexokinase assay) and the LC/MS-determined abundance of glucose
and kynurenine are shown for the first and validation cohort.

| | Median Glucose, | | Median Glucose | | Median Kynurenine, | |
|---|---|---|---|---|---|---|
| | mq/dL (IQR) | | (IQR) | | (IQR) | |
| First Cohort | | | | | | |
| Non-Mucinous (n = 6) | 82 | (66-105) | 516,398 | (104,255-844,052) | 195,686 | (185,655-565,007) |
| SCA (n = 4) | 86 | (67-162) | 690,415 | (516,398-1,423,682) | 189,236 | (113,892-378,913) |
| Pseudocyst (n = | 61 | (25-96) | 175,405 | (104,255-246,555) | 3,210,834 | (198,553-6,223,115) |
| Mucinous (n = 20) | 5 | (5-18) | 22,875 | (7,885-122,083) | 12,954 | (1,376-53,566) |
| MCN (n = 4) | 7 | (5-19) | 55,432 | (15,269-154,376) | 36,830 | (11,424-53,092) |
| IPMN (n = 6) | 5 | (5-5) | 7,784 | (1,102-32,264) | 3,949 | (1-7,882) |
| Cancer (n = 10) | 16 | (5-38) | 27,202 | (10,113-155,926) | 46,805 | (1,376-90,310) |
| Validation Cohort | | | | | | |
| Non-Mucinous (n = 8) | 58 | (20-130) | 151,709 | (55,034-265,430) | 95,660 | (46,531-143,193) |
| SCA (n = 4) | 103 | (58-157) | 214,618 | (151,709-369,758) | 124,564 | (75,152-179,259) |
| Pseudocyst (n = | 20 | (13-82) | 55,034 | (43,748-56,275) | 64,978 | (46,531-114,289) |
| Mucinous (n = 11) | 5 | (5-21) | 31,204 | (3,187-56,275) | 1,435 | (1-6,825) |
| MCN (n = 1) | 16 | | 43,551 | | 6,825 | |
| IPMN (n = 8) | 5 | (5-8) | 19,923 | (2,882-43,740) | 967 | (1-1,471) |
| Cancer (n = 2) | 23 | (21-25) | 73,304 | (33,836-112,771) | 12,348 | (3,029-21,666) |

Metabolomic Analysis

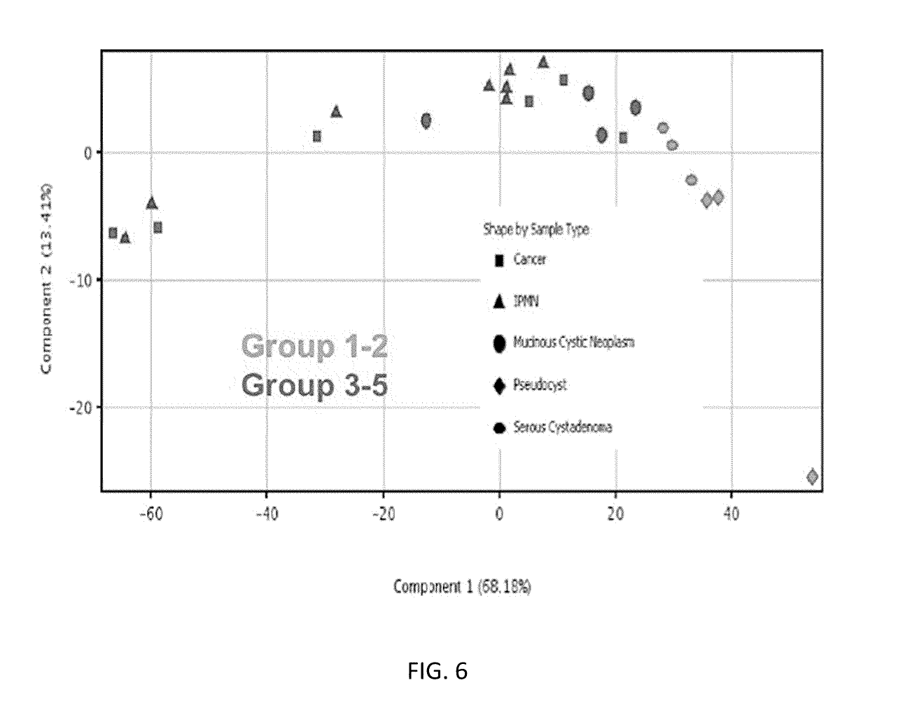

A total of 506 metabolites were detected in the first cohort. Principal component analysis indicated that non-mucinous (SCA and PC) and mucinous (MCN, IPMN, and cancer) cysts could be separated based upon the measured metabolite abundances (FIG. 6). Mucinous cysts could not be separated out from those cysts harboring cancer. Among the total detected metabolites, 10 were differentially abundant in the mucinous and non-mucinous cysts, using a threshold cutoff of a fold-change >2.0 and p-value <0.05. Four of these 10 metabolites were also differentially abundant in the validation cohort, and the identities of 2 of these were determined to be glucose and kynurenine (Table 3). The remaining eight metabolites could not be matched to any known metabolite, and their abundance was very low. Despite several attempts to identify them by MS/MS analysis, we could not obtain a sufficient amount to enable their characterization.

TABLE 3
Differentially abundant metabolites in mucinous and non-mucinous pancreatic
cyst fluids obtained from two independent clinical cohorts. The accurate mass,
retention time (RT), metabolite abundance (+standard error of the mean),
fold change (FC), and p-value (calculated using a two-sample t-test with unequal
variance on log-transformed data) for the four metabolites that were differentially
abundant in the two cohorts are shown. The abundances of the two un-identified
metabolites were too low to enable their identification.

| | | | | | | |
|---|---|---|---|---|---|---|
| Metabolite | Mass | RT | FC | Mucinous | Non-Mucinous | P value |

First Cohort (n = 26 samples)

| | | | | | | |
|---|---|---|---|---|---|---|
| Glucose | 413.1159 | 7.257 | −2.2 | 96716 + 39743 | 625669 + 168585 | 0.015 |
| Kynurenine | 441.1352 | 11.234 | −24.8 | 30115 + 8656 | 483315 + 330377 | 0.002 |
| Unknown #1 | 772.2558 | 13.243 | −30.1 | 4782 + 1962 | 23161 + 11469 | 0.033 |
| Unknown #2 | 726.2774 | 13.035 | −174.4 | 2919 + 1447 | 34255 + 15871 | 0.007 |

Validation Cohort (n = 19 samples)

| | | | | | | |
|---|---|---|---|---|---|---|
| Glucose | 413.1143 | 7.257 | −7.2 | 47439 ± 17800 | 161759 ± 37245 | 0.004 |
| Kynurenine | 441.1356 | 11.159 | −179.8 | 20881 ± 17295 | 109825 ± 21233 | 0.002 |
| Unknown #1 | 772.2560 | 12.558 | −808.1 | 1550 ± 1300 | 12221 ± 5579 | 8 × 10⁻⁵ |
| Unknown #2 | 726.2774 | 13.035 | −505.4 | 2870 ± 799 | 4430 ± 1729 | 5 × 10⁻⁵ |

Reduced Glucose Levels in Mucinous Cysts

Table 2 shows that in the first and validation cohorts, the glucose abundance as measured by LC/MS analysis, was significantly reduced in mucinous relative to non-mucinous cysts (p=0.0001 and p=0.005, respectively). The area under the receiver operator curve (ROC) was 0.92 (95% CI 0.81-1.00) and 0.88 (95% CI 0.72-1.00) in the first and validation cohorts (FIG. 1).

To confirm these observations, a spectrophotometric hexokinase assay for glucose (used at Stanford clinical laboratory) was used to measure cyst glucose levels (Kunst et al., supra). The median (interquartile range, IQR) cyst glucose level in mucinous cysts [5 mg/dL (5-18)] was over 16-fold below that in non-mucinous cysts [82 mg/dL (66-105)] in the first cohort (p=0.002) (Table 2). In the validation cohort, the median cyst glucose level in mucinous cysts [5 mg/dL (5-21)] was 10-fold below that in non-mucinous cysts [58 mg/dL (20-130)] (p=0.01) (Table 2). Cyst fluid glucose levels could not differentiate mucinous pre-malignant cysts from cancerous cysts.

Combining data from both cohorts, the ROC was 0.88 (95% CI 0.76-0.99). The highest diagnostic accuracy was observed using a cutoff of 66 mg/dL for differentiating non-mucinous from mucinous cysts. With this threshold, cyst fluid glucose had a sensitivity and specificity of 94% and 64%, respectively, for classifying mucinous and non-mucinous cysts (FIG. 2).

Serous Cystadenomas have Elevated Glucose Levels

Among non-mucinous cysts, the median glucose levels of SCAs were higher than PCs in both cohorts. When combining the cohorts, the median (IQR) cyst glucose level of SCAs (n=8) was 98 mg/dL (67-157 mg/dL) compared to PCs (n=6) that was 23 mg/dL (20-96 mg/dL) (p=0.07). When cyst glucose levels of SCAs were compared to all non-SCAs (PC, IPMN, MCN, and cancer) the median cyst glucose level was significantly elevated (98 mg/dL versus 7 mg/dL) (p=0.0001) with a ROC curve of 0.93 (95% CI 0.86-1.0). The highest diagnostic accuracy was obtained at a cutoff of 66 mg/dL with a sensitivity and specificity for differentiating SCA from non-SCA lesions of 88% and 89% respectively.

Glucose Performs Similarly to CEA in Differentiating Mucinous from Non-Mucinous Cysts

Since cyst fluid CEA data was available for 31 of the 45 samples when combining the cohorts, we could compare the relative diagnostic performance of cyst fluid glucose and CEA as diagnostic markers for differentiating mucinous from nonmucinous cysts. The median (IQR) CEA levels in non-mucinous (n=9) and mucinous (n=22) cysts were 1.7 ng/ml (0.9-69) and 985 ng/ml (173-5797) respectively, which was significantly different (p=0.0005). Among the 22 mucinous cysts, the median CEA level for pre-malignant cysts (n=15) was 319 ng/ml (IQR: 171-5797), which was not significantly different (p=0.323) from malignant cysts (n=7) (median 2298 ng/ml, IQR: 319-11962). Using the standard cutoff of 192 ng/ml, CEA had a diagnostic accuracy, sensitivity, and specificity of 77%, 73%, and 89% respectively. In this sample, glucose, at a cutoff of <66 mg/dL, had a diagnostic accuracy, sensitivity, and specificity for diagnosing mucinous from non-mucinous cysts of 84%, 95%, and 56%, respectively. Requiring both CEA>192 ng/ml AND glucose <66 mg/dl as combined criteria for differentiating mucinous from non-mucinous cysts did not improve the diagnostic accuracy (74%) relative to either marker alone. Using either CEA>192 ng/ml OR glucose <66 mg/dL to differentiate mucinous from non-mucinous cysts showed a trend of improved diagnostic accuracy (87%) compared to CEA or glucose alone, but this was not statistically significant.

Mucinous Cysts have Reduced Kynurenine Abundance

In the first cohort, the kynurenine abundance in the mucinous cysts (median: 12,954) was significantly reduced (p=0.0006) relative to benign non-mucinous cysts (median: 195,686). In the validation cohort, the kynurenine abundance in mucinous cysts (median: 1,435) was also significantly below (p=0.002) that in non-mucinous cysts (median: 95,660) (Table 2). Differences in extraction efficiency and detection sensitivity for each LC/MS run used to evaluate metabolite levels lead to different absolute abundance levels observed in the 2 different cohorts. No significant difference was observed between mucinous pre-malignant cysts and cancerous cysts.

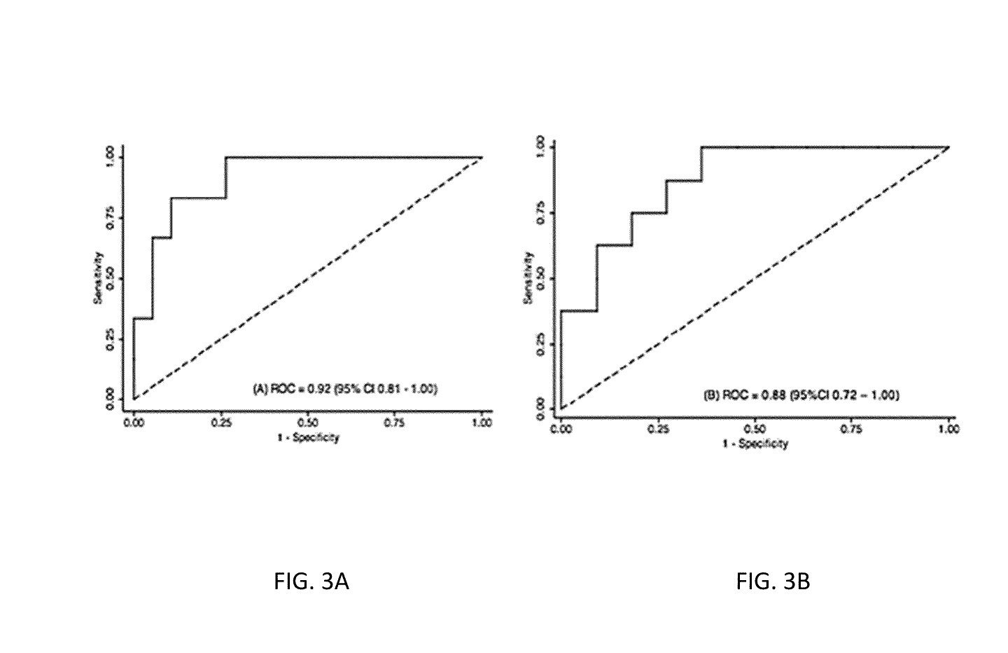

Data from each cohort were separately analyzed to evaluate the performance of kynurenine as an indicator of whether a cyst was mucinous or non-mucinous. The ROC for kynurenine was 0.94 (95% CI 0.81-1.00) and 0.92 (95% CI 0.76-1.00) in the first and validation cohorts (FIG. 3). In the first cohort, the maximum diagnostic accuracy was observed at a cutoff abundance of 185,650 providing a sensitivity and specificity of 100% and 80%, respectively. In the validation cohort, an abundance level of 34,000 provided the maximum diagnostic accuracy providing a sensitivity and specificity of 90%, and 100%, respectively. In the absence of an established assay for kynurenine, direct comparison with CEA and glucose was not performed at this time.

Serous Cystadenomas have Elevated Kynurenine Abundance

For the purposes of distinguishing SCA lesions among all non-SCA lesions, the kynurenine abundance was compared in both cohorts. In the first cohort, SCA lesions had a significant kynurenine abundance (median (IQR) 189,236 (113,892-378,913)) compared to non-SCA lesions (median (IQR) 21,043 (1,805-79,297)) (p=0.038). In the validation cohort, SCA lesions also had a significant kynurenine abundance compared to non-SCA lesions (median (IQR) 124,564 (75,152-179,259) versus 3,029 (732-54,862) (p=0.035). The area under the ROC curve was 0.83 (95% CI 0.63-1.0) and 0.85 (95% CI 0.66-1.0) for the first and validation cohorts respectively.

Discussion

With increasing recognition of the prevalence of pancreatic cysts and the pre-malignant potential in a substantial proportion of them, better diagnostic tools are needed. In recent years, there has been growing interest in cyst fluid based biomarkers with reports of potential clinical utility using DNA, RNA, and cytokine expression profiling methods (Ke et al. (2009) Pancreas 38(2):e33-42; Wu et al. (2011) Sci. Transl. Med. July 20; 3(92):92ra66; Ryu et al. (2011) Pancreatology 11(3):343-350; Allen et al. (2009) Ann. Surg. 250(5):754-760; Khalid et al. (2009) Gastrointest. Endosc. 69(6):1095-1102. In this study we describe the potential clinical utility of metabolite profiling for identifying novel pancreatic cyst fluid biomarkers.

Metabolomic profiling for the identification of disease biomarkers in serum has had very limited success, which is (at least in part) due to the effects of diet and other confounding factors, as well as the ultra-complex pattern of metabolites present in serum. In this study we focused on pancreatic cyst fluid, which is a relatively isolated space, and hypothesized that tissues immediately surrounding the cyst may have a relatively stronger effect on the metabolites present in the cyst fluid. Using a semi-targeted approach, we identified glucose and kynurenine as metabolites that were differentially abundant by clinically relevant cyst categories in two independent cohorts. The identity of glucose and kynurenine was confirmed by MS/MS analysis and by comparison to chemical standards. Since glucose is a commonly measured analyte, we were able to rapidly validate the observations of our metabolomic profile using a widely available assay found in most clinical laboratories. Cyst fluid glucose levels were significantly decreased in mucinous cysts compared to non-mucinous cysts providing a diagnostic accuracy, sensitivity, and specificity of 84%, 94%, and 64%, respectively, when using a threshold value of 66 mg/dL. Of further clinical relevance, SCA lesions were uniquely elevated when compared to the other cyst types. Cyst fluid glucose could differentiate SCA from non-SCA lesions with a diagnostic accuracy, sensitivity, and specificity of 89%, 88%, and 89% respectively, when using a similar threshold of 66 mg/dL.

Based on the diagnostic performance of glucose, we compared it to CEA—the one biomarker currently accepted and widely used in clinical practice to differentiate mucinous from non-mucinous cysts. CEA performed similarly in differentiating mucinous from non-mucinous cysts to that reported in the literature (Brugge et al. (2004) Gastroenterology 126(5):1330-1336; van der Waaij et al. (2005) Gastrointest. Endosc. 62(3):383-389). Glucose had a similar diagnostic accuracy compared to CEA (84% versus 77%). Using either CEA>192 ng/ml or glucose <66 mg/dL to differentiate mucinous from non-mucinous cysts did not significantly improve the diagnostic accuracy (87%) compared to CEA or glucose alone.

These observations of glucose in pancreatic cysts are clinically meaningful and warrant further validation. Analyzing glucose required a very small amount of cyst fluid (50 μL) and it was done rapidly in our hospital laboratory. In contrast, CEA analysis for many centers requires sending 300-500 μL of cyst fluid out to a reference laboratory with a consequent delay in results. Further, the high diagnostic accuracy of glucose for SCA lesions may minimize the number of patients who require imaging surveillance for indeterminate pancreatic cysts.

Kynurenine plays an important role in pancreatic cancer and immune biology so it was of great interest to observe a differential abundance between mucinous and non-mucinous cysts (Chen et al. (2009) Int. J. Tryptophan Res. 2:1-19; Opitz et al. (2011) Nature 478(7368):197-203; Witkiewicz et al. (2008) J. Am. Coll. Surg. 206(5):849-854, discussion 854-856; Vander Heiden (2011) Nat. Rev. Drug Discov. 10(9):671-684; Vander Heiden et al. Cold Spring Harb. Symp. Quant. Biol. 2012 Jan. 19). We observed decreased kynurenine abundances associated with mucinous cysts compared to non-mucinous cysts in 2 independent cohorts with a diagnostic accuracy of approximately 95%. Similar to glucose, we also observed that kynurenine abundances were significantly elevated in SCA lesions when compared to non-SCA lesions. Further analysis combining glucose and CEA with kynurenine was not performed at this time due to the lack of an available standardized assay for kynurenine.

There are several limitations that should be considered when evaluating the results of this study. A significant limitation includes the relatively small sample size of each cohort, which limits our ability to consider potential confounding factors and correctly identify other real differences. Furthermore, this study did not include less common types of pancreatic cysts, such as cystic neuroendocrine tumors. To ensure a clear gold standard, this study only included patients with a histological diagnosis. The vast majority of individuals underwent surgery because of cysts with recognized high-risk features, which could also introduce a bias in this cohort that differ from the larger population of individuals with pancreatic cysts.

Cyst fluid used for this analysis was acquired during surgery in 14 (54%) of 26 cases in the first cohort and 15 (79%) of 19 cases in the validation cohort. The remaining cases had cyst fluid collected pre-operatively by EUS. The different method of cyst fluid acquisition theoretically may influence the metabolite results. We compared CEA and glucose levels between surgically collected and EUS collected samples by cyst category and did not observe a significant difference. A recent study comparing EUS and surgery collected cyst fluid also observed no difference (Partyka et al. (2012) J. Proteome Res. 11(5):2904-2911).

In this study, we defined cancer to include mucinous cysts with high-grade dysplasia and carcinoma. Among invasive carcinomas, we included cases where it was not clear whether the cyst was a consequence of tumor degeneration or malignant transformation of a mucinous cyst. Although the biology may be different between these two types of cysts, we chose to include them because differentiating them in clinical practice can be difficult. We did not observe a significant difference in glucose levels between presumed IPMN cancers and adenocarcinomas with cystic degeneration.

While metabolomic profiling may shed insight into the pathophysiology of pancreatic cysts, it does not provide a mechanism for differential metabolite expression. Individual hypotheses regarding the mechanism for different glucose and kynurenine levels between the different types of cysts exist based on current understanding of pancreatic tumor biology, and warrant further investigation. The potential of metabolomic profiling to identify other biomarkers remains as the semi-targeted derivatization method used here only labels a restricted set of metabolites with certain chemical features (primary and secondary amines and a few other functionalities). Other labeling methods could be used to identify other metabolomic markers, particularly those that differentiate mucinous pre-malignant from cancerous cysts.

In conclusion, we used a novel metabolomic profiling approach on 2 separate histologically defined pancreatic cyst cohorts and discovered glucose and kynurenine to have promise as clinically useful cyst biomarkers. While they may differentiate mucinous from non-mucinous cysts, they may actually be a more specific biomarker for serous cystadenomas. Such a biomarker would have significant clinical utility.

While the preferred embodiments of the invention have been illustrated and described, it will be appreciated that various changes can be made therein without departing from the spirit and scope of the invention.

What is claimed is:

1. A method for distinguishing mucinous and non-mucinous cysts, the method comprising:
a) obtaining a sample of pancreatic cyst fluid from a subject;
b) measuring the levels of one or more biomarkers in the pancreatic cyst fluid, wherein the one or more biomarkers are selected from the group consisting of glucose and kynurenine; and
c) analyzing the levels of one or more biomarkers in conjunction with respective reference levels for the biomarkers, wherein similarity of the levels of one or more biomarkers in the cyst fluid to reference value levels for a mucinous cyst indicates that the cyst in the subject is a mucinous cyst, and wherein similarity of the levels of one or more biomarkers in the cyst fluid to reference levels for a non-mucinous cyst indicates that the cyst in the subject is a non-mucinous cyst.

2. The method of claim 1, comprising measuring the level of glucose and kynurenine.

3. The method of claim 1, wherein a level of glucose greater than or equal to 66 mg/dL indicates that the pancreatic cyst is a non-mucinous pancreatic cyst.

4. The method of claim 1, wherein a level of glucose less than 66 mg/dL indicates that the pancreatic cyst is a mucinous pancreatic cyst.

5. The method of claim 1, wherein a lower level of kynurenine compared to the level of kynureinine in pancreatic cyst fluid from one or more benign non-mucinous cysts indicates that the pancreatic cyst is a mucinous pancreatic cyst.

6. The method of claim 1, further comprising measuring the level of amphiregulin.

7. The method of claim 6, wherein a level of amphiregulin greater than 300 pg/ml indicates that the pancreatic cyst is a malignant mucinous pancreatic cyst.

8. The method of claim 1, wherein the levels of one or more biomarkers are correlated with the type of mucinous or non-mucinous pancreatic cyst.

9. The method of claim 8, wherein the pancreatic cyst is a pseudocyst, a serous cystadenoma, a mucinous cystic neoplasm, or an intraductal papillary mucinous neoplasm.

10. The method of claim 6, wherein the levels of one or more biomarkers are correlated with malignant potential of the pancreatic cyst.

11. The method of claim 1, further comprising distinguishing a pseudocyst from a serous cystadenoma.

12. The method of claim 1, further comprising distinguishing a serous cystadenoma from a non-serous cystadenoma.

13. The method of claim 1, wherein the subject is a human being.

14. The method of claim 1, wherein the pancreatic cyst fluid sample is obtained by endoscopic ultrasound fine-needle aspiration.

15. The method of claim 1, wherein measuring the amount of one or more biomarkers in the pancreatic cyst fluid comprises performing mass spectrometry, an enzymatic or biochemical assay, liquid chromatography, NMR, an enzyme-linked immunosorbent assay (ELISA), a radioimmunoassay (RIA), an immunofluorescent assay (IFA), or a Western Blot.

16. A method for determining the malignant potential of a pancreatic cyst in a subject, the method comprising:
a) obtaining a sample of pancreatic cyst fluid from a subject,
b) measuring the amount of amphiregulin in the pancreatic cyst fluid derived from the subject, and
c) analyzing the amount of amphiregulin in conjunction with reference levels for amphiregulin, wherein the reference levels are determined by analyzing the amounts of amphiregulin in pancreatic cyst fluid samples derived from subjects with malignant mucinous pancreatic cysts, wherein the amount of amphiregulin in the pancreatic cyst fluid sample is correlated with the malignant potential of the pancreatic cyst.

17. The method of claim 16, wherein a level of amphiregulin greater than 300 pg/ml indicates that the subject has pancreatic cancer or high-grade dysplasia.

18. A method of monitoring a pancreatic cyst in a subject, the method comprising:
a) analyzing a first pancreatic cyst fluid sample from a subject to determine the levels of one or more biomarkers, wherein the one or more biomarkers are selected from the group consisting of glucose, kynurenine, and amphiregulin, wherein the first sample is obtained from the subject at a first time point;
b) analyzing a second pancreatic cyst fluid sample from the subject to determine the levels of the one or more biomarkers, wherein the second sample is obtained from the subject at a second time point; and
c) comparing the levels of the one or more biomarkers in the first pancreatic cyst fluid sample to the levels of the one or more biomarkers in the second pancreatic cyst fluid sample in order to detect any changes in the status of the pancreatic cyst in the subject over time.

19. The method of claim 18, wherein a level of glucose greater than or equal to 66 mg/dL indicates that the pancreatic cyst is a non-mucinous pancreatic cyst.

20. The method of claim 18, wherein a level of glucose less than 66 mg/dL indicates that the pancreatic cyst is a mucinous pancreatic cyst.

21. The method of claim 18, wherein a level of amphiregulin greater than 300 pg/ml indicates that the subject has pancreatic cancer or high-grade dysplasia.

22. The method of claim 21, further comprising comparing the level of amphiregulin in pancreatic cyst fluid samples from the subject to reference levels for amphiregulin for high grade dysplasia, cancer in situ, and invasive cancer.

23. A method for treating a pancreatic cyst in a subject, the method comprising: obtaining a sample of pancreatic cyst fluid from the pancreatic cyst in the subject, and surgically removing the pancreatic cyst from the subject if the level of amphiregulin in the pancreatic cyst fluid sample is greater than 300 pg/ml.

24. The method of claim 23, wherein the pancreatic cyst fluid sample is obtained by endoscopic ultrasound fine-needle aspiration.

25. The method of claim 23, wherein the amphiregulin is measured with an immunoassay.

26. A method for determining the prognosis of a subject who has a pancreatic cyst, the method comprising:
a) obtaining a sample of pancreatic cyst fluid from the subject,
b) measuring the amount of glucose in the pancreatic cyst fluid derived from the subject, wherein a level of glucose greater than or equal to 66 mg/dL indicates that the subject is at low risk of developing pancreatic cancer; and
c) measuring the amount of amphiregulin in the pancreatic cyst fluid derived from the subject, wherein a level of amphiregulin greater than 300 pg/ml indicates that the subject is at high risk of developing pancreatic cancer.

27. A method for monitoring the efficacy of a therapy for treating pancreatic cancer or dysplasia in a subject, the method comprising: analyzing the levels of amphiregulin in pancreatic cyst fluid samples derived from the subject before and after the subject undergoes said therapy, in conjunction with respective reference levels for amphiregulin.

28. The method of claim 27, wherein increasing levels of amphiregulin in the subject indicate that the condition of the subject is worsening and decreasing levels of amphiregulin in the subject indicate that the condition of the subject is improving.

29. The method of claim 27, wherein the level of amphiregulin in pancreatic cyst fluid samples from the subject is compared to reference levels for amphiregulin for high grade dysplasia, cancer in situ, and invasive cancer to determine the stage of disease progression.

30. The method of claim 27, further comprising analyzing the level of glucose or kynurenine in pancreatic cyst fluid samples derived from the subject before and after the subject undergoes said therapy, in conjunction with respective reference levels for glucose or kynurenine.

31. A biomarker panel for diagnosing pancreatic cysts comprising one or more biomarkers selected from the group consisting of glucose, kynurenine, and amphiregulin.

32. The biomarker panel of claim 31 comprising glucose and kynurenine.

33. The biomarker panel of claim 32, further comprising amphiregulin.

34. A kit comprising agents for measuring the levels of one or more biomarkers in pancreatic cyst fluid from a subject, wherein the one or more biomarkers are selected from the group consisting of glucose, kynurenine, and amphiregulin; and instructions for using the kit to diagnose pancreatic cysts.

35. The kit of claim 34, further comprising one or more control reference samples.

36. The kit of claim 34, further comprising information, in electronic or paper form, comprising instructions to correlate the detected levels of glucose, kynurenine, or amphiregulin with the type of pancreatic cyst present.

37. The kit of claim 34, further comprising reagents for performing an immunoassay to detect amphiregulin.

38. The kit of claim 37, wherein the agents comprise at least one antibody that specifically binds to amphiregulin.

39. The kit of claim 34, further comprising reagents for performing a hexokinase-glucose-6-phosphate dehydrogenase coupled enzyme assay for detecting glucose.
